(19) United States
(12) Patent Application Publication
(10) Pub. No.: US 2012/0128620 A1
(43) Pub. Date: May 24, 2012

(54) ANIMAL LITTER

(76) Inventor: Larry Douglas Mohr, Tempe, AZ (US)

(21) Appl. No.: 13/304,156

(22) Filed: Nov. 23, 2011

Related U.S. Application Data
(60) Provisional application No. 61/417,077, filed on Nov. 24, 2010.
(60) Provisional application No. 61/490,517, filed on May 26, 2011.

(51) Int. Cl. A61L 11/00; A01K 1/01; A61L 9/01; A01K 29/00

(52) U.S. Cl. 424/76.6; 119/171

(57) ABSTRACT

An animal litter is provided. The animal litter includes zeolite, a sub-blend comprising organic proteins, amino acids, carbohydrates, surfactants, and oxidizers, and a blend of bacteria that functions as a biological inhibitor, wherein the zeolite and the sub-blend are coupled together by the application of the blend of bacteria. Under the condition that animal waste comes into contact with the animal litter, the zeolite absorbs the liquid in the animal waste, the blend of bacteria destroys parasites and bacteria within the animal waste as well as constructs a physical barrier around the animal waste to inhibit the spread of the parasites and bacteria outside the physical barrier, and the sub-blend controls and neutralizes the odors emanating from the animal waste.

### CROSS REFERENCE TO RELATED APPLICATION[S]

This application claims priority to U.S. Provisional Patent Application to Mohr entitled “ANIMAL LITTER,” Ser. No. 61/417,077, filed Nov. 24, 2010, and to U.S. Provisional Patent Application to Mohr entitled “ANIMAL LITTER,” Ser. No. 61/490,517 filed May 26, 2011, the disclosures of which are hereby incorporated entirely herein by reference.

### BACKGROUND OF THE INVENTION

1. Technical Field

This invention relates generally to improvements in animal sanitation, and in particular to an improved animal litter.

2. State of the Art

Pet owners have long been concerned with the health concerns associated with the collection and disposal of animal waste. Pet owners that have “indoor” pets are particularly concerned because of the relatively close proximity of the pet's waste to the pet owner, not to mention the fact that the pet owner must physically remove the pet's waste from the pet owner's dwelling from time to time. Pets that live predominantly indoors usually have a place designated by the pet owner in which the pet can deposit its waste products. If left unattended to for some time, the odors may permeate throughout the pet owner's dwelling.

In the case of cats, a litter box is usually the pet owner's means for the efficient collection and disposal of the cat's waste. Litter is typically placed within an appropriately-sized container (e.g., litter box) in which the cat, overtime, becomes accustomed to using. After the pet uses the litter box, the waste solids are generally buried within the litter by the pet and the waste liquid is absorbed by the litter. The soiled litter is thereafter periodically separated by the pet owner from the remaining litter and discarded. The litter must also be replenished or completely replaced over time and the litter box must be cleaned.

Because of the requirement to remove the pet's waste product from the litter and the need to clean the cat litter box itself, pet owners frequently come into close proximity and even into immediate contact with the pet's waste. Appropriately, many of the health worries surrounding ownership of cats is centered on the cat litter box and the cat's fecal matter.

Certain bacteria and parasites are commonly found in animal waste, including cat waste, the bacteria and parasites including Toxoplasmosis, Escherichia coli (“E. coli”), and Campylobacter jejuni, among others. Toxoplasmosis is an infection caused by a tiny parasite called Toxoplasma gondii which can be found in dirty cat litter boxes and outdoor soil where cat feces can be found. According to the Center for Disease Control and Prevention (CDC) more than 60 million people in the United States may be infected with the Toxoplasma parasite. Fortunately, very few people ever experience any symptoms because a healthy person's immune system usually keeps the parasite from causing illness. However, pregnant women and individuals who have compromised immune systems, such as individuals infected with the HIV virus, are at risk and should take precautions to avoid being infected by the parasite. For people in this group a Toxoplasma infection could cause serious health problems to the individual or to a pregnant woman's unborn child.

Similar to Toxoplasmosis, E. coli can also infect humans through contact with feline fecal material. E. Coli is a bacterium commonly found in the intestinal tract of both humans and animals, a few strains of which can produce powerful toxins and cause severe illness, especially in children under 5 years of age.

Also, the genus of bacteria known as Campylobacter is found commonly in a wide variety of healthy domestic and wild animals, including cats. The bacteria usually live in the intestines as part of the animal's normal flora, and is shed in the feces. Campylobacter jejuni is now the leading cause of bacterial gastroenteritis, otherwise known as gastrointestinal illness or food poisoning. Campylobacter infections are contagious, especially among members of the same family and children in day care or preschools.

In view of the above, there is a need in the pet industry to provide an animal litter that not only effectively inhibits the spread of illness by bacteria and parasites commonly found in animal waste but also eliminates the foul odors stemming from animal litter.

### SUMMARY OF THE INVENTION

The present invention relates to improvements in animal sanitation, and in particular to an improved animal litter that not only prohibits the spread of illness through contact with animal waste but also eliminates the accompanying odors of the waste.

An aspect of the animal litter includes zeolite, a sub-blend comprising organic proteins, amino acids, carbohydrates, surfactants, and oxidizers, and a blend of bacteria that functions as a biological inhibitor, wherein the zeolite and the sub-blend are coupled together by the application of a binder and the blend of bacteria is applied to the binded zeolite and sub-blend combination.

In addition, under the condition that the animal waste comes into contact with the animal litter described above, the zeolite absorbs the liquid in the animal waste, the blend of bacteria destroys parasites and bacteria within the animal waste as well as constructs a physical barrier around the animal waste that inhibits the spread of the parasites and bacteria outside of the physical barrier, and the sub-blend controls and neutralizes the odors emanating from the animal waste, both in the waste and in the surround air around the waste.

Another aspect of the animal litter includes a method of manufacturing the animal litter, comprising combining a combination of different bacteria with water, mixing zeolite and a sub-blend, the sub-blend comprising organic proteins, amino acids, carbohydrates, surfactants, and oxidizers, and applying the combination of different bacteria with water onto the blended zeolite and sub-blend, wherein during the application the water activates a binder within the sub-blend to adhere the sub-blend to the zeolite and the bacteria adheres to the combined zeolite and sub-blend to create the animal litter.

Another aspect of the animal litter includes a method of controlling animal waste using the animal litter, comprising combining a combination of different bacteria with water, mixing zeolite and a sub-blend, the sub-blend comprising organic proteins, amino acids, carbohydrates, surfactants, and oxidizers, applying the combination of different bacteria with water onto the blended zeolite and sub-blend, wherein the bacteria adheres to the combined zeolite and sub-blend to create the animal litter; and controlling animal waste deposited into the animal litter, wherein the animal litter absorbs liquid in the animal waste, destroys parasites and bacteria within the animal waste, constructs a physical barrier around the animal waste to inhibit the spread of parasites and bacteria outside of the physical barrier, and neutralizes the odors emanating from the animal waste.

In an alternative embodiment, Psyllium Husk Powder is added to the composition of animal litter described above to allow the animal litter composition to clump in response to the application of water contained in the liquid waste of the animal.

The foregoing and other features and advantages of the present invention will be apparent from the following more detailed description of the particular embodiments of the invention.

### DETAILED DESCRIPTION OF EMBODIMENTS

As discussed above, embodiments of the present invention relate to improvements in animal sanitation, and in particular to an improved animal litter that not only prohibits the spread of illness through contact with animal waste but also eliminates the accompanying odors of the waste.

The animal litter comprises a natural-occurring, porous-structured material, such as for example, zeolite, which is a volcanically deposited, hydrated aluminosilicate that possesses an open three-dimensional crystalline framework structure. Specifically, zeolite has a unique interconnecting lattice structure arranged to form a honeycomb framework of interconnecting channels and pores. Negatively-charged alumina building blocks and neutrally-charged silica building blocks are stacked, thereby producing an open, three-dimensional honeycomb framework. Such structure is characterized by pronounced macro and micro porosity that gives rise to unique absorption, catalytic, and ion-exchange properties that form the basis of a wide range of uses. Moreover, the ability of zeolite to be surface modified by ion exchange, to have non-ionic surfactants applied thereto, and to be loaded internally with proteins and amino acids provides endless options for uses in remediation of odor and disease control. In fact, zeolite provides the base material of the animal litter of the present invention.

The zeolite used in the animal litter of the present invention is a low-sodium, low dust, high cation exchange capacity (CEC) material. Of course, the higher the CEC of the zeolite, the greater the amount of positive charged materials that can be held to the surface of the zeolite. The zeolite of the present invention carries at least twice the CEC of that of conventional cat litters made from clay or diatomaceous earth (e.g., diatomite). The zeolite of the present invention carries a CEC of between 150 to 200 meq/100 gm, as compared with the CEC of the diatomaceous earth, 50 meq/100 gm, or clay, 25 meq/100 gm, of conventional litters. The zeolite used in the animal litter is classified to a sieve size of 14 to 40, or in other words, the zeolite particles fit through a mesh opening measuring approximately between 0.4 mm and 1.4 mm.

The animal litter further comprises a sub-blend of ingredients, wherein the sub-blend comprises: one of two oxidizing compounds (such as Trichlormelamine (TCM) or Sodium Percarbonate); amino-acids (such as L-glutamine, cysteine, Inositol, and glycolic acid); proteins (such as Soy protein and Whey protein); surfactants (such as Sodium dodecylbenzene sulfonate); and a carbohydrate (such as simple sugars (e.g., monosaccharide)). In certain embodiments, the carbohydrate acts as the binder to bind the sub-blend to the base zeolite, upon the application of liquid to the carbohydrate. In addition to the ingredients above, the sub-blend may further include nitrogen converters and digesters, such as, Lactobacillus plantarum; Lactobacillus jensenii; Lactobacillus lactis; Lactobacillus delbruckii; Lactobacillus salviarius; Leuconostoc salvairius; Bifido bacterium, Nitrosomas, Nitrobacter, Azotobacter, and Rhizobium.

The animal litter also comprises a blend of a selected group of bacteria that function as biological inhibitors. Combining these certain bacteria in specific quantities produces a class of bio-enzymes that act as biological inhibitors. These biological inhibitors beat out pathogenic bacteria, usually found within animal waste, in the race for the needed nutrients required for the pathogenic bacteria to grow and replicate. Thus, the biological inhibitors, or bio-enzymes produced by the selected group of bacteria, literally starve the pathogenic bacteria to death by consuming the available needed nutrients before the pathogenic bacteria can do so. Moreover, these biological inhibitors produce a zone or perimeter around themselves that enzymatically deters other bacteria from entering therein. It follows that the greater the number of biological inhibitors present, the less opportunity and availability there is for pathogens to grow and survive.

The selected blend of bacteria that operate as described above includes seven different bacteria that function synergistically as biological inhibitors, the bacteria including Bacillus licheniformis, Bacillus amyliquefaciens, Bacillus cereus, Bacillus pumilus, Bacillus subtilis, Bacillus spherious, and Bacillus pumilus. These biological inhibitors are blended with reverse osmosis, deionized (RO/DI) water, the resulting colony forming unit (CFU) of blended bacteria not exceeding four (4) billion per bacteria type. This mixture of bacteria and RO/DI water is applied to the base zeolite and the sub-blend that have been mixed together. Application of the liquid mixture serves a two-fold purpose. First, the RO/DI water is a vehicle for the bacteria to transport and deposit the bacteria on the zeolite and sub-blend. Second, the water activates the binder in the sub-blend to bind the zeolite, the sub-blend, and the bacteria together to form the animal litter.

The above-described blend of bacteria in this liquid mixture is in a non-vegetative state. However, in response to liquid waste of the animal being deposited into the animal litter, these bacteria become active. When active, they establish a comprehensive network of communication, wherein the various types of bacteria communicate with one another in an enzymatic language regarding the liquid waste. Specifically, the enzymatic language includes the specific bacteria types producing a signal-producing protein, molecule, or chemical that these bacteria types expel and use to communicate with surrounding bacteria. The surrounding bacteria receive the signal-producing protein, molecule, or chemical at a receptor site and process the signal. The signal-producing protein, molecule, or chemical can be intra-species or inter-species, thus each bacteria type can announce itself to bacteria of the same species as well as to bacteria of differing species. By producing signals and receiving signals in this way, these bacteria types communicate with one another and to determine how many of the surrounding bacteria are of the same species. Taking an accounting of numbers allows bacteria species to behave collectively, or to at least coordinate collective behavior, once a satisfactory number of the specific bacteria has been accounted for, or otherwise accumulated. Thus, depending on the signal received, certain bacteria types may remain de-active until future notice while others may activate and initiate pathogenicity. Further, certain bacteria may deactivate if their number decreases below certain levels. It follows then that such deactivation or activation can depend on the number of bacteria currently present in the host organism. In summary, the bacteria strands create enzymes in response to the liquid waste coming into contact with the animal litter, and, because of the relative close proximity of the various bacteria types with one another in the blend of bacteria, the enzymes communicate with one another in the enzymatic language to activate or deactivate as needed, which can aid in the destruction and inhibition of specific pathogenic bacteria and parasites. For example, but not by way of limitation, the blend of bacteria may activate, grow, and thereby inhibit the growth of the pathogenic bacteria, wherein the pathogenic bacteria is eliminated or shuts down due to the lack of numbers, or comparative numbers with respect to the blend of bacteria.

The cat litter is manufactured by combining together the zeolite, the sub-blend of ingredients, and the blend of bacteria. The zeolite and the sub-blend are first combined in a mixer, such as a mechanical blender. After the zeolite and the sub-blend are mixed and combined, the blend of bacteria, which has previously been mixed with RO/DI water, is sprayed, or otherwise distributed, onto the zeolite and sub-blend. In some embodiments, the blend of bacteria is sprayed onto the zeolite and the sub-blend as the zeolite and the sub-blend are being mixed. In some embodiments, 12 gallons of RO/ID water containing the blend of bacteria, is sprayed onto one (1) ton of mixed zeolite and sub-blend. As stated, the liquid application of the blend of bacteria provides two functions, which are: (1) the liquid application activates a binder in the sub-blend that adheres the sub-blend components to the zeolite and reduces dust, and (2) the liquid application applies the blend of bacteria, or the biological inhibitors, to both the zeolite and the sub-blend ingredients.

As mentioned above, the resulting animal litter inhibits the spread of pathogenic bacteria in animal waste as well as controls the resulting odor of the waste. The function of the biological inhibitors is to destroy, or at least effectively inhibit, the spread and growth of pathogenic bacteria deposited into the animal litter through the animals' solid waste products, whereas the function of the sub-blend is to react with the urine and solid waste of the animal to control the associated odor. The animal litter provides these functions in response to coming into contact with animal waste. Indeed, the animal litter is designed and structured to react to, and combat, the negative implications of both liquid and solid animal waste. Specifically with regard to cats, solid waste contains parasites and other undesirable bacteria, whereas liquid waste contains urea, organic nitrogenous compounds, and fatty acids.

As mentioned, the animal litter reacts to both the solid and liquid waste. In response to the liquid waste, specifically urine, the surfactant in the sub-blend is activated and aids to absorb the liquid into the matrix of the zeolite. However, the absorption of the liquid does not result in the animal litter clumping. In other words, the composition of the animal litter results in the liquid being absorbed without the animal litter forming into a “scoopable,” i.e., removable, compact mass. In addition, the amino acids in the sub-blend aid in the conversion of ammonia (NH₃) to ammonium (NH₄), with L-glutamine, inositol, and glycolic acid being mainly responsible for this conversion and stabilization of nitrogen. The pungent ammonia is thus protonated by the animal litter, resulting in odorless ammonium that is stable and effectively charge-bound to the zeolite. Indeed, due to the structural configuration of the animal litter, over 90% of the aromatic ammonia is converted or absorbed by the reaction with the bacteria, amino acids, and the zeolite.

With regard to the solid waste, the zeolite itself functions as a dehydrator of the waste. With the help of sodium found in zeolite, the liquid in the solid waste is extracted, or absorbed, from the waste into the matrix of the zeolite. The sodium helps break down the surface tension between the liquid in the solid waste and the surface of the zeolite, thus allowing the liquid to be absorbed by the large surface area of the zeolite created by the extensive lattice structure. Once dehydrated, the odors emanating from the solid waste are significantly, if not completely reduced.

In addition, once the solid waste comes into contact with the animal litter, the biological inhibitors in the blend of bacteria activate, begin to communicate, and begin to inhibit the spread of parasites and other unwanted bacteria found in the solid waste. In doing so, the animal litter not only destroys the parasites and other undesirable bacteria contained in the solid waste but also creates a physical barrier around the perimeter of the solid waste that inhibits the spread of such unwanted organisms beyond the waste itself. Specifically, once activated, the biological inhibitors in the blend of bacteria beat out the pathogenic bacteria in the animal waste in the race for nutrients. Without the needed nutrients required for the pathogenic bacteria to grow and replicate, the pathogenic bacteria die. Thus, the biological inhibitors, or bio-enzymes produced by the selected group of bacteria, literally starve the pathogenic bacteria to death by consuming the available needed nutrients before the pathogenic bacteria can do so. Also, by beating out the pathogenic bacteria in the race for nutrients, the biological inhibitors grow and multiply at a greater rate than the pathogenic bacteria and form a physical barrier around the animal waste. As a result, the animal litter both destroys the pathogenic bacteria and parasites and creates a physical barrier that prevents these parasites and unwanted bacteria from spreading to either humans that must periodically come into close proximity of the waste or to other animals that must use the same litter box. In certain embodiments, the animal litter destroys the parasites and bacteria within the animal waste by applying anti-parasitic agents capable of inhibiting or even curing infections. Cysteine and inositol are two amino acids that are potent anti-parasitic agents.

In addition to that described above, the structural configuration of the animal litter allows bio-vapors containing the bio-enzymes in the animal litter to be released into the air from the zeolite in response to a rise in the ambient temperature and/or a rise in the relative humidity. Specifically, the ability of the zeolite to “off-gas” allows the bio-enzymes to release from the animal litter as vapor and enter the surrounding air. These enzymes in the bio-vapor function as an air-born odor control to reduce the number of organic malodors in the air around the litter box on contact. The enzymes in the bio-vapor are the same enzymes that are triggered when the solid and liquid waste come into contact with the animal litter and provide like function.

The animal litter is thus able to prohibit the spread of illness through contact with animal waste but also eliminate the accompanying odors of the waste not only in the waste itself but also in the surrounding air.

In an alternative embodiment, Psyllium Husk Powder is added to the composition of animal litter described above. The Psyllium Husk Powder may bind to the zeolite and sub-blend by activation of the binder upon application of the liquid blend of bacteria. The presence of the Psyllium Husk Powder allows the animal litter composition to clump in response to the application of liquid contained in the waste of the animal. In other words, by adding Psyllium Husk Powder to the animal litter described above, the Psyllium Husk Powder will react with the liquid contained in the waste of the animal and form the litter into compact masses of litter that are capable of being removed entirely from the remaining un-clumped litter. Specifically, by applying the Psyllium Husk Powder to the composition of animal litter described above at an application rate of between 3.0-5.0 grams of between 70-99% pure Pysillium Husk Powder to between 400-500 grams of the zeolite-based animal litter described above, the resulting composition of litter will clump in response to the application of liquid contained in the animal waste. Further, additional embodiments of the zeolite-based animal litter described above combined with the Psyllium Husk Powder are comprised of between 3.5-4.5 grams of 80-90% pure Pysillium Husk Powder applied to between 425-475 grams of the zeolite-based animal litter. Further yet, additional embodiments of the clumping animal litter are comprised of about 4.0 grams of 80% pure Pysillium Husk Powder applied to approximately 445-450 grams of the zeolite-based animal litter.

Psyllium Husk Powder has been used for many years as a digestive aid in both animals and humans. The Psyllium Husk Powder contained in the clumping animal litter composition described above provides advantages over conventional clumping animal litters. For example, conventional litters often use sodium bentonite clay to create a clumping effect. However, it is well-established that sodium bentonite clay causes health risks and concerns, especially when ingested or inhaled by animal or human alike. Specifically, the powerful clumping abilities of sodium betonite cause the ingested clay dust and particles to combine with natural and ingested liquid to form a solid mass within the animal or human. Moreover, when the ingested or inhaled bentonite comes into contact with liquid, the betonite has the capacity to swell to approximately 15 times its original volume, thus resulting in possible dehydration and internal swelling. In contrast to these health problems, the relatively low formulated rates of Psyllium Husk Powder used to create the clumping litter of the present disclosure causes no such ill effects. Moreover, there are health benefits associated with the ingestion of Psyllium Husk Powder, such as improved digestion due in part to Psyllium Husk's relatively high fiber count compared with other grains. The Psyllium Husk Powder may help regulate the digestive system of animals that suffer from constipation or diarrhea. In addition, other reasons for the use of Psyllium Husk Powder in the clumping animal litter composition of the present disclosure are: (1) it is an organic natural plant fiber; and (2) the small quantities used in this blending option have no adverse health issues if ingested or inhaled. It follows that if and when animals ingest or inhale the animal litter of the present disclosure, such as when cats lick their paws and intake the animal litter thereon, the Psyllium Husk Powder could aid the digestive system of the animal.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. An animal litter, comprising:
a porous base material;
a sub-blend comprising an organic protein, an amino acid, a carbohydrate, a surfactant, an oxidizer, and a binder; and
a blend of bacteria in a liquid mix;
wherein under the condition that the sub-blend is mixed with the porous base material and the liquid mix is applied thereto, the liquid mix activates the binder to couple together the porous base material, the sub-blend, and the blend of bacteria to form the animal litter.

2. The animal litter of claim 1, wherein the porous base material is zeolite having a cation-exchange capacity of 150 to 200 meq/100 gm and a sieve size of 14 to 40.

3. The animal litter of claim 1, wherein the blend of bacteria comprises one or more bacteria types, the bacteria types including Bacillus licheniformis, Bacillus amyliquefaciens, Bacillus cereus, Bacillus pumilus, Bacillus subtilis, Bacillus spherious, and Bacillus pumilus.

4. The animal litter of claim 3, wherein the blend of bacteria is blended with reverse osmosis, deionized (RO/DI) water to form a colony forming unit, the resulting colony forming unit (CFU) not exceeding four billion per bacteria type.

5. The animal litter of claim 1, wherein the organic protein comprises soy protein and whey protein, wherein the amino acid comprises L-glutamine, cysteine, Inositol, and glycolic acid, wherein the carbohydrate comprises a monosaccharide, wherein the surfactant comprises sodium dodechylbenzene sulfonate, and wherein the oxidizing compound comprises one of Trichlormelamine (TCM) and Sodium Percarbonate.

6. The animal litter of claim 1, wherein the sub-blend further comprises nitrogen converters and digesters.

7. The animal litter of claim 6, the converters and digesters comprising Lactobacillus plantarum, Lactobacillus jensenii, Lactobacillus lactis, Lactobacillus delbruckii, Lactobacillus salviarius, Leuconostoc salvairius, Bifido bacterium, Nitrosomas, Nitrobacter, Azotobacter, and Rhizobium.

8. The animal litter of claim 1, wherein the structure of the animal litter and the amino acids in the sub-blend are adapted to convert over 90% of ammonia (NH₃) in animal waste to ammonium (NH₄).

9. The animal litter of claim 1, wherein under the condition that animal waste comes into contact with the animal litter, the porous base material absorbs liquid from the animal waste, the blend of bacteria reacts to the animal waste and destroys parasites and bacteria in the animal waste and constructs a physical barrier around the animal waste that inhibits the spread of parasites and bacteria from within the animal waste to beyond the physical barrier, and the sub-blend controls malodors emanating from the animal waste.

10. The animal litter of claim 1, further comprising 70-99% pure Pysillium Husk powder, wherein 3.0 to 5.0 grams of Psyllium Husk powder is applied to 400 to 500 grams of the animal litter.

11. The animal litter of claim 10, wherein under the condition that the powder comes into contact with the liquid from the animal waste the powder forms the animal litter that comes into contact with the liquid from the animal waste into a clump that is removable from remaining portions of the animal litter.

12. A method of manufacturing an animal litter, comprising:
combining a blend of bacteria with water;
mixing a porous base material and a sub-blend, the sub-blend comprising organic proteins, amino acids, carbohydrates, surfactants, and oxidizers; and
applying the blend of bacteria with water to the mixed porous base material and sub-blend; and
coupling the porous base material, the sub-blend, and the blend of bacteria together to create the animal litter.

13. The animal litter of claim 12, wherein the porous base material comprises zeolite having a cation-exchange capacity of 150 to 200 meq/100 gm and a sieve size of 14 to 40

14. The animal litter of claim 12, wherein the blend of bacteria comprises one or more bacteria types, the bacteria types including Bacillus licheniformis, Bacillus amyliquefaciens, Bacillus cereus, Bacillus pumilus, Bacillus subtilis, Bacillus spherious, and Bacillus pumilus, and the water comprises reverse-osmosis, deionized (RO/DI) water.

15. The animal litter of claim 14, wherein the blend of bacteria and water forms a colony forming unit, the resulting colony forming unit (CFU) not exceeding four billion per bacteria type.

16. The animal litter of claim 12, wherein the organic proteins comprise soy protein and whey protein, wherein the amino acids comprise L-glutamine, cysteine, Inositol, and glycolic acid, wherein the carbohydrates comprise simple sugars, wherein the surfactants comprise sodium dodechylbenzene sulfonate, and wherein the oxidizing compounds comprise one of Trichlormelamine (TCM) and Sodium Percarbonate.

17. The animal litter of claim 12, further comprising activating a binder in the sub-blend by the application of the blend of bacteria with water to the mixed porous base material and sub-blend, the binder thereby coupling the porous base material, the sub-blend, and the blend of bacteria together to create the animal litter.

18. A method of controlling animal waste using an animal litter, the method comprising:
providing an animal litter comprising a porous base material, a sub-blend, and a blend of bacteria;
absorbing liquid into the porous base material from animal waste that comes into contact with the animal litter;
destroying parasites and bacteria within the animal waste by the blend of bacteria reacting with the animal waste;
constructing a physical barrier around the animal waste by the blend of bacteria reacting with the animal waste, the physical barrier inhibiting the spread of parasites and bacteria from within the animal waste to outside the physical barrier; and
neutralizing malodors emanating from the animal waste by the sub-blend reacting to the animal litter.

19. The method of claim 18, wherein the neutralizing malodors emanating from the animal waste further comprises:
off-gassing bio-enzymes from the animal litter into air surrounding the animal litter;
neutralizing malodor vapors emanating from the animal waste by the bio-enzymes reacting with the malodor vapors.

20. The method of claim 18, wherein the destroying parasites and bacteria within the animal waste further comprises:
combining bacteria in specific quantities in the animal litter that react to animal waste to produce bio-enzymes that are configured to outcompete pathogenic bacteria in the animal waste in the race for the needed nutrients required for the pathogenic bacteria to grow and replicate, thus starving and destroying the parasites and pathogenic bacteria in the animal waste.
